(19) United States
(12) Patent Application Publication
(10) Pub. No.: US 2025/0391547 A1
(43) Pub. Date: Dec. 25, 2025

(54) Medical Billing Classification Prediction

(71) Applicant: Cerner Innovation, Inc., Kansas City, MO (US)

(72) Inventors: Monica Gaur, Delhi (IN); Rupanjali Chaudhuri, Bangalore (IN); Suman Pal, Bangalore (IN)

(73) Assignee: Cerner Innovation, Inc., Kansas City, MO (US)

(21) Appl. No.: 18/749,953

(22) Filed: Jun. 21, 2024

(51) Int. Cl. G16H 40/20; G06F 40/284; G16H 10/60

(52) U.S. Cl. CPC G16H 40/20; G06F 40/284; G16H 10/60

(57) ABSTRACT

Techniques for early prediction of medical billing classification codes and associated medical billing costs using routine clinical text are disclosed. The system predicts the medical billing codes within defined hours of admission by generating vector embeddings from a set of medical notation data, bypassing the need for post-discharge medical codes. Using a novel segmentation technique, the system processes lengthy medical notation data by dividing them into smaller subsequences. These subsequences are input to a large language model (LLM) to generate a plurality of sets of probability values for a set of medical billing classifications. The system selects a particular predicted medical billing classification for the patient based on the sets of probability values. Additionally, the system estimates medical billing costs early in the admission process. The system ensures comprehensive context utilization from clinical notes, enabling hospitals to manage treatment expenses proactively and improve operational efficiency.

### TECHNICAL FIELD

The present disclosure relates to predicting medical billing codes and corresponding medical billing. In particular, the present disclosure relates to applying machine learning models to treatment data to predict a medical billing classification code for a patient.

### BACKGROUND

Inpatient care is the medical care extended to patients whose condition requires admission to the hospital. The Inpatient Prospective Payment System (IPPS) categorizes each inpatient hospital admission with similar clinical and treatment characteristics into a Diagnosis-Related Group (DRG), where patients in the same group are expected to incur a similar cost from hospital resource utilization. According to the Centers for Medicare and Medicaid Services (CMS), each DRG has a fixed payment rate based on the average cost of resources used to treat a specific diagnosis category. The DRGs were developed to enable an effective framework that would improve the efficiency of procedures and treatments for patients with the same disease category, thereby standardizing the costs without degrading the quality of care given to the patient.

The DRGs are a patient classification scheme that provides a means of relating the type of patients a hospital treats reflected as a case mix to the costs incurred by the hospital. The introduction of DRGs in prospective payment systems has put pressure on hospitals to optimize cost and quality with efficient resource utilization. DRG-based statistics are reviewed by hospital managers to assess its patient mix and financial efficiency under DRG reimbursement. Hospitals allocate experts for the manual calculation of DRG. This is a time-consuming process. Since DRGs are conventionally obtained post-discharge, this makes it impossible for hospitals to act upon such vital information about DRG and potential spending on care for active patients and claim a reimbursement in case of over-spending. Hence, hospitals need a streamlined process that requires accurate coding and could aid in improving cost estimates and resource allocation.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIG. 2 illustrates an example set of operations for predicting medical billing classification codes for medical services in accordance with one or more embodiments;

FIGS. 3A and 3B illustrate an example set of operations for training a machine learning model to generate probability values for a set of medical billing classification codes;

FIGS. 4A and 4B illustrate an example embodiment; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. MEDICAL BILLING CLASSIFICATION CODE PREDICTION ARCHITECTURE
3. PREDICTING MEDICAL BILLING CLASSIFICATION CODES FOR MEDICAL SERVICES
4. TRAINING A MACHINE LEARNING MODEL TO GENERATE PROBABILITY VALUES FOR MEDICAL BILLING CLASSIFICATION CODES
5. EXAMPLE EMBODIMENT
6. COMPUTER NETWORKS AND CLOUD NETWORKS
7. MICROSERVICE APPLICATIONS
8. HARDWARE OVERVIEW
9. MISCELLANEOUS; EXTENSIONS

### 1. General Overview

One or more embodiments apply a machine-learning model to pre-discharge medical notation information to predict medical billing classification codes for patients. The medical notation data may include, for example, physician notes from a physician's discussion with a patient or a physician's diagnosis for the patient. The medical billing classification codes may be, for example, Diagnosis-Related Groups (DRGs). The system partitions the medical notation data into multiple sequences. The system applies the trained machine learning model to the sequences to generate probabilities for medical billing classification codes for the sequences. The system selects a predicted medical billing classification code for the patient from among the medical billing classification codes for the sequences.

According to an example embodiment, for each sequence in a set of multiple sequences associated with a patient, the system may generate a set of probability values for a corresponding set of medical billing classification codes. The system selects a particular predicted medical billing classification code for the patient based on the probability values. For example, the system may select as a predicted medical billing classification code for the patient, the medical billing classification code associated with the highest mean probability value across the set of sequences, a highest cumulative probability value, a highest median probability value, a highest overall probability value, or any combination of multiple criteria.

One or more embodiments prepare training datasets for training the machine learning model. The system determines data input parameters of the model, including a feature input limit and feature types that the model can receive, as input data to generate predictions. The token input limit represents a maximum number of tokens the machine learning model may ingest as input data. For example, a particular type of machine learning model may be capable of receiving input values for no more than 450 features. However, a set of medical notation data may correspond to 4,000 tokens. The tokens may include both words that are included in medical notation data and sub-words generated based on words in the medical notation data. The sub-words include sets of letters that are less than a full word. For example, if the medical notation data includes the word “alzheimers,” the system may generate four additional sub-words: “al,” “##z,” “##hiemer,” and “##heimers,” where the #symbol represents “any letter.” If the number of tokens resulting from the words and sub-words in a set of medical notation data exceeds the token input limit, the system generates multiple sequences of tokens as input data. In addition, if a model requires a minimum number of feature values as input data, the system may generate additional sub-words from among the words in the medical notation data to meet the minimum threshold of feature values. The system applies the machine learning model to each sequence to generate medical billing classification code predictions and probability values for the sequence.

One or more embodiments described in this Specification and/or recited in the claims may not be included in this General Overview section.

### 2. Medical Billing Classification Code Prediction Architecture

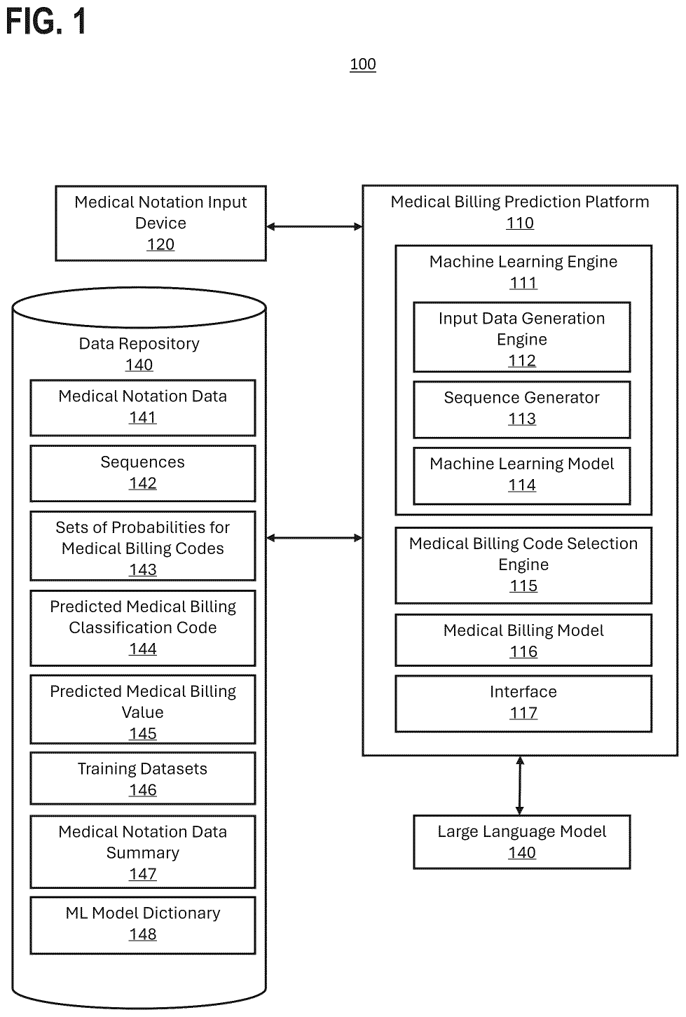

FIG. 1 illustrates a system 100 in accordance with one or more embodiments. As illustrated in FIG. 1, system 100 includes a medical billing prediction platform 110, a medical notation input device 120, a large language model 130, and a data repository 140. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Components may be distributed over multiple applications and/or machines. Multiple components may be combined into one application and/or machine. Operations described with respect to one component may instead be performed by another component.

Additional embodiments and/or examples relating to computer networks are described below in Section 5, titled “Computer Networks and Cloud Networks.”

The medical billing prediction platform 110 includes one or more computers, such as servers, in communication with one or more medical notation input devices 120. For example, a medical notation input device 120 may be a medical practitioner's office computer, tablet computer, or handheld device. The medical notation input device 120 may be a computer capable of receiving voice notes and transcribing them into text.

The medical billing prediction platform 110 receives medical notation data via an interface 117. The interface may include a program that transmits graphical user interface (GUI) data to the medical notation input device 120. Alternatively, the interface may include a set of protocols for communicating with the medical billing prediction platform 110, storing medical notation data 141 in a data repository 140, and retrieving stored medical notation data 141.

In one or more embodiments, interface 117 refers to hardware and/or software configured to facilitate communications between a user and the medical billing prediction platform 110. Interface 117 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of interface 117 are specified in different languages. The behavior of user interface elements is specified in a dynamic programming language such as JavaScript. The content of user interface elements is specified in a markup language, such as hypertext markup language (HTML) or XML User Interface Language (XUL). The layout of user interface elements is specified in a style sheet language such as Cascading Style Sheets (CSS). Alternatively, interface 117 is specified in one or more other languages, such as Java, C, or C++.

In one or more embodiments, a data repository 140 is any type of storage unit and/or device (e.g., a file system, database, collection of tables, or any other storage mechanism) for storing data. Furthermore, a data repository 140 may include multiple different storage units and/or devices. The multiple different storage units and/or devices may or may not be of the same type or located at the same physical site. Furthermore, a data repository 140 may be implemented or executed on the same computing system as the medical billing prediction platform 110. Additionally, or alternatively, a data repository 140 may be implemented or executed on a computing system separate from the medical billing prediction platform 110. The data repository 140 may be communicatively coupled to the medical billing prediction platform 110 via a direct connection or via a network.

Information describing medical notation data 141, sequences 142, sets of probabilities 143 for medical billing classification codes, predicted medical billing classification codes 144, predicted medical billing values 145, machine learning model training datasets 146, and medical notation data summaries 147 may be implemented across any of components within the system 100. However, this information is illustrated within the data repository 140 for purposes of clarity and explanation.

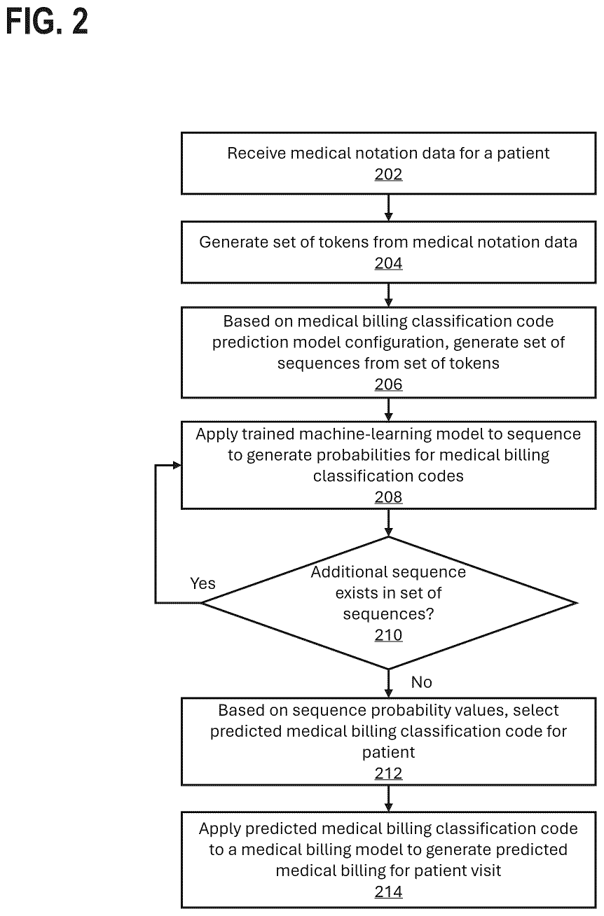

In one or more embodiments, the medical billing prediction platform 110 refers to hardware and/or software configured to perform operations described herein for generating medical billing classification code predictions. Examples of operations for generating medical billing classification code predictions are described below with reference to FIG. 2. While the embodiments in FIG. 1 and FIG. 2 describe an architecture and operations for generating medical billing classification code predictions, embodiments are not limited to medical billing classification codes. Instead, embodiments encompass any medical billing classification codes that may be used to generate and/or predict medical billing values for patients.

In an embodiment, the medical billing prediction platform 110 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

The medical billing prediction platform 110 is communicatively coupled with a large language model (LLM) 130. Large language models are a type of deep learning model that combines a deep learning technique, called attention, with a deep learning model type, known as transformers, to build predictive models. These predictive models encode and predict natural language writing. LLMs contain hundreds of billions of parameters trained on multiple terabytes of text. LLMs are trained to receive natural language as an input. LLMs typically generate natural language as an output. In addition, some LLMs may be trained to output computer code, visual output (such as images), and audio output. LLMs are made up of layers of attention mechanisms and neural networks that process input data in parallel. The layers of attention mechanisms and neural networks operating in parallel allow the LLM to learn complex patterns in text.

The attention mechanisms help neural networks to learn the context of words in the sequences of words. An attention mechanism operates by breaking down a set of input data, such as a sentence or sequence of words or tokens, into keys, queries, and values. Keys represent elements of the input data that provide information about what to pay attention to. Queries represent elements of the input data that need to be compared with the keys to determine relevance. Values are elements of the input data that will be selected or weighted based on the attention scores. The attention mechanism calculates a similarity score between each query and key pair. This score reflects how relevant each key is to a given query. Various methods can be used to compute these scores, such as dot-product, scaled dot-product, or other custom functions. The similarity scores are then transformed into attention weights. For example, a system may transform the similarity scores using a softmax function. The softmax function adjusts the values of the similarity scores relative to each other, so the sum of the similarity scores is 1. Finally, the attention weights are used to take a weighted sum of the corresponding values. This weighted sum represents the model's focused or “attended” representation of the input data. In one or more embodiments, the attention mechanisms are implemented using self-attention processes, scaled dot-product attention processes, and multi-head attention processes.

In operation, the LLM receives a natural language prompt as input data and generates a sequence of words in natural language by predicting a next word, or sequence of words, based on the textual and grammatical patterns learned by the LLM during training. In the example embodiment of FIG. 1, the medical billing prediction platform 110 provides medical notation data 141 as a text prompt to the LLM 130. The medical notation data includes text words, codes, abbreviations, and alphanumeric content. The LLM 130 generates a medical notation data summary 147. The medical notation data summary 147 may include natural language content. The medical notation data summary 147 may exclude codes, abbreviations, and alphanumeric content that was included in the medical notation data 141 and that do not include natural language content.

The machine learning engine 111 includes an input data generation engine 112. The input data generation engine 112 prepares the medical notation data 141 for input to the machine learning model 114. The input data generation engine 112 cleans and processes the text content of the medical notation data 141. For example, the input data generation engine 112 converts text into lowercase, deletes particular patterns (such as **2156-10-21**) from the text, deletes stop words from the text, separates alphanumeric text with a space, and replaces two or more continuous spaces with a single space.

The input data generation engine 112 separates the cleaned text content into tokens. A token is a set of one or more characters that are grouped together. For example, a token may be a word separated from adjacent tokens by spaces. In addition, a number (e.g., “30) is a separate token. The system may identify tokens within the medical notation data 141 using a tokenizer. The tokenizer determines if a character is a part of a token associated with an adjacent, previously analyzed character, or if the character is part of a separate token. The tokenizer may consider attributes of the characters to determine if two adjacent characters belong to the same token. In addition, the input data generation engine 112 may analyze additional attributes of the text to identify semantic meaning of the text (such as emphasis) and a relatedness of tokens to each other. For example, the input data generation engine 112 may analyze the following: a font of the characters, an amount of spacing or distance between characters, spacing associated with the font, a formatting style of the characters, a language of the characters, and a character type (e.g., alphanumeric or punctuation) of the characters.

In one or more embodiments, the input data generation engine 112 determines whether or not to generate sub-words from tokens generated from the medical notation data 141 by comparing the tokens to a machine learning model dictionary 148. The dictionary 148 includes a mapping of known tokens to embeddings. In one example, the ML model dictionary 148 specifies an integer ID for each word in the dictionary 148. The input data generation engine 112 uses the integer IDs to look up embeddings for the tokens. If the input data generation engine 112 determines that a particular token is not in the dictionary 148, the input data generation engine divides the token into sub-words. For example, the input data generation engine 112 may determine the word “alzheimers” is not in the dictionary 148. As a result, the input data generation engine 112 divides the word “alzheimers” into sub-words: “al,” “##z,” “##hiemer,” and “##heimers,” where the #symbol represents “any letter.”

A sequence generator 113 generates multiple sequences 142 of tokens based on configuration data of the machine learning model 114. For example, the sequence generator 113 may determine that the medical billing classification prediction machine learning model receives as input data N values corresponding to N tokens. The sequence generator 113 generates M sequences 142 by dividing a total number of tokens (corresponding to words and sub-words) T by N, where T/N=M, if the number of features N divides evenly into the number of tokens T, and T/N=M−1, if the number of features N divides into the number of tokens T with a remainder. For example, if the number of tokens that a medical billing classification prediction machine learning model is configured to receive is N=512, then the system generates one sequence if T is less than or equal to 512, two sequences if T is greater than 512 and less than or equal to 1024, etc.

In one embodiment, the sequence generator 113 selects a number of sequences 142 and a size of the sequences 142 based on machine learning model 114 configuration data, such as the size and type of input data the machine learning model 114 is configured to receive. Additionally, or alternatively, the sequence generator 113 may generate a number of tokens or modify a number of generated tokens based on the machine learning model 114 configuration data. For example, if the number of features N divides into the number of tokens T with a remainder, the sequence generator 113 may generate a number of additional sub-words equal to the remainder. Alternatively, if the remainder is below a threshold, the sequence generator 113 may remove a number of sub-words equal to the remainder from the set of tokens.

The medical billing prediction platform 110 provides each sequence 142 generated by the sequence generator 113 to the machine learning model 114 to generate sets of probabilities 143 for a set of medical billing classification codes. For each sequence, the machine learning model 114 generates a plurality of probability values corresponding to a respective plurality of medical billing classification codes. For example, the machine learning model may be trained to generate 10 output values that correspond to a predefined set of 10 medical billing classification codes. The 10 medical billing classification codes may be identified as the most frequently occurring medical billing classification codes. While a set of 10 medical billing classification codes is provided above as an example, embodiments encompass any number of output values, such as 15, 20, or 25 that correspond to different medical billing classification codes.

In one embodiment, the machine learning model 114 includes a foundational model trained on a broad text corpus and a separately trained classification head comprising one or more neural network layers. The foundational model is trained on the dataset, including a broad text corpus, to generate an embedding corresponding to an input sequence. The parameters of the foundation model are frozen, the classification head is added to an output of the foundation model, and the classification head is trained on narrower training datasets 146 than the foundational model. The narrower training datasets 146 include sequences of tokens and medical billing classification codes assigned to the sequences. The trained model, including the foundation model and classification head, generates probability values for multiple medical billing classification codes based on the embedding generated by the foundation model.

In one or more embodiments, the foundation model is an encoder-only type model that does not include a decoder. For example, the foundation model may be a Bidirectional Encoder Representations from Transformers (BERT) type model or a ClinicalBERT type model. ClinicalBERT is a BERT type model that is further trained on clinical notes. In a BERT type model, a transformer is an attention mechanism that specifies a level of weight (“attention”) a model should give to a particular token. The transformer recognizes contextual relationships between words in a set of text. The “bidirectionality” of the BERT model refers to how the transformer obtains context information for a particular token in a sequence from both a preceding token in the sequence and a subsequent token.

In one or more embodiments, the machine learning engine 111 implements a machine learning algorithm that can be iterated to train the machine learning model 114 that best maps a set of input variables to an output variable using a set of training data. In particular, the machine learning algorithm is configured to generate and/or train the fine-tuned machine learning model 114, including a pre-trained foundation model and the fine-tuning classification head.

In one or more embodiments, the large language model (LLM) 130 is a decoder-only type model such as a generative pre-trained transformer (GPT) type model. The LLM 130 generates a set of output text based on an input prompt. In the embodiment illustrated in FIG. 1, the input prompt includes medical notation data 141. The output text includes a natural language summary 147 of the medical notation data 141. The output from the decoder-only type LLM 130 is converted into tokens and sequences of tokens. The sequences are provided as input data into the encoder-only type machine learning model 114. While the LLM 130 is configured to generate text based on input prompts, the foundation model of the machine learning model 114 is configured to generate embeddings from input text. The classification head of the machine learning model generates probability values for a set of medical billing classification codes based on the embeddings.

A predicted medical billing classification code selection engine 115 selects a medical billing classification code as the predicted medical billing classification code associated with a set of medical notation data 141 and a corresponding set of sequences 142 of tokens. The predicted medical billing classification code selection engine 115 analyzes multiple sets of probability values. The multiple sets of probability values are respectively associated with a set of medical billing classification codes. For example, the machine learning model 114 generates a first set of probability values for a set of medical billing classification codes based on a first sequence of tokens generated based on the medical notation data 141. The machine learning model 114 generates a second set of probability values for the same set of medical billing classification codes based on a second sequence of tokens generated based on the same medical notation data 141. The machine learning model 114 may generate a third set of probability values for the same set of medical billing classification codes based on a third sequence of tokens generated based on the same medical notation data 141. The medical billing classification code selection engine 115 applies a mathematical or logical algorithm to the sets of probability values for the set of sequences associated with the medical notation data 141 to select a particular predicted medical billing classification code for the medical notation data 141. The mathematical or logical algorithm may include one or more of the following: the highest mean probability calculated based on the probability values across the set of sequences, the highest median probability value, the highest overall probability value, and a probability value that is above a first threshold value across a number of sequences that meets a second threshold number of sequences. In one or more embodiments, the medical billing classification code selection engine 115 may apply a weight to one or more probability values prior to selecting the predicted medical billing classification code. For example, different sequences may be associated with different sets of source data, such as a patient intake survey, vitals measurements, initial physician observations, initial treatment observations, and a physician diagnosis. The medical billing classification code selection engine 115 may apply a higher weight to a sequence associated with a physician's diagnosis than with an initial patient intake survey. In one embodiment, the medical billing classification code selection engine 115 applies a graduated weight scale to the sequences in which sequences corresponding to older medical notation data 141, such as data generated closer to the patient intake, are assigned a lower weight than data generated later in the patient's treatment. Based on applying the mathematical and/or logical algorithm to the sets of probabilities 143, the medical billing classification code selection engine 115 selects a particular medical billing classification code 144 as the predicted medical billing classification code 144 for the set of medical notation data 141 associated with a particular patient's visit to a particular healthcare provider.

In one or more embodiments, the medical billing classification code selection engine 115 selects a set of two or more medical billing classification codes as the predicted medical billing classification codes for the medical notation data 141. For example, the medical billing classification code selection engine 115 may apply an algorithm to select, as predicted medical billing classification codes for a set of medical notation data 141, a set of medical billing classification codes (a) having the highest mean probability values and (b) whose mean probability values across a set of sequences add up to at least 0.8. According to another example, the medical billing classification code selection engine 115 may select, as a predicted medical billing classification group, the three medical billing classification codes having the highest mean probability values across a set of sequences.

A medical billing model 116 generates a predicted medical billing value for a patient visit based on the predicted medical billing classification code 144 or a set of predicted medical billing classification codes. According to one embodiment, the model maps the medical billing classification codes to different medical billing weights. The medical billing model 116 calculates the predicted medical billing value for the patient's visit by multiplying the medical billing weight associated with the predicted medical billing classification code 144 by a standardized amount associated with the healthcare provider generating the medical notation data 141. The medical billing model 116 may further apply additional adjustment factors to generate the predicted medical billing value 145. Adjustment factors may include, for example, cost of living adjustment based on a geographic location of a healthcare provider, a patient's age, a number of diagnoses, a predicted length of stay, and whether or not a healthcare provider is a teaching hospital.

### 3. Predicting Medical Billing Classification Codes for Medical Services

FIG. 2 illustrates an example set of operations for predicting medical billing classification codes for medical services in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

In an embodiment, the system obtains medical notation data for a patient (Operation 202). Medical notation data includes data obtained from patient-provided forms, such as a check-in form, patient interactions with staff and medical personnel to describe and/or demonstrate symptoms, and patient interactions with physicians. In one embodiment, the system obtains medical notation data for a defined period of time. The defined period of time may be prior to a discharge of a patient. For example, a patient's entire experience at a hospital may include (a) entering the emergency room, (b) describing symptoms, (c) being admitted to the hospital, (d) being treated in an intensive care unit (ICU), (e) being monitored in a longer-term care unit of the hospital, and (f) being discharged from the hospital. Events (a)-(d) may occur within the first 24 hours of the patient being admitted. Event (e) may occur after 24 hours. Event (f) may occur after 48 hours. Each event may be associated with a set of medical notations, including medical staff observations, tests, and diagnoses. The system may obtain the medical notation data once 24 hours have elapsed from the time the patient was admitted to the hospital. Accordingly, medical notation data associated with events (a)-(d) may be available. Medical notation data associated with events (e) and (f) may be neither generated nor available. According to another example, the predefined time may be 48 hours from the time the patient was admitted to the hospital. Accordingly, the medical notation data for events (a)-(e) may be available. The medical notation data for event (f) may be neither generated nor available.

The system generates a set of tokens from the medical notation data (Operation 204). Generating the set of tokens includes pre-processing or cleaning text content and generating additional tokens. Text cleaning includes eliminating or modifying data that is inaccurate, unnecessary, duplicative, or structured incorrectly. Pre-processing the text includes converting text into lowercase, removing patterns in the text that are identified as unnecessary (such as “**2156-10-21**”), removing stop words (such as a, I, the, in, of, for, etc.), separating alphanumeric text with a space, and replacing two or more continuous spaces with a single space.

The system generates the set of tokens by dividing the cleaned text into words and/or word parts. The system identifies words that are to be identified as tokens without modification to the words and other words that are to be divided into sub-parts to generate multiple tokens from the word. For example, the system may identify the words “old,” “65,” and “patient” as tokens without dividing the words into sub-parts. The system may divide the word “alzheimers” into sub-words “al,” “##z,” “##hiemer,” and “##heimers,” where the #symbol represents “any letter.” The system may determine words that are to be divided into multiple sub-words based on comparing the words from the medical notation data with a predefined dictionary of words associated with a trained machine learning model. If a word in the medical notation data is not among the dictionary of words, the system divides the word into sub-words. If the word in the medical notation data is included among the dictionary of words, the system does not divide the word into sub-words.

According to one example, the trained machine learning model includes a natural language processing (NLP) machine learning model. The NLP model maps tokens (i.e., words and sub-words) to embedding vectors that are fed into the model. Accordingly, a word in the medical notation data that is not among the dictionary of words is a word for which there is no existing mapping to an embedding vector. The system generates sub-words for these words. In some examples, the sub-words include symbols representing the sub-word's position within a word. For example, a #symbol prior to the sub-word indicates the sub-word is not at the front of the word. A #symbol following the sub-word indicates the sub-word is not at the end of the word.

The system generates a set of sequences from the set of tokens corresponding to the medical notation data (Operation 206). In one or more embodiments, the system generates the set of sequences based on machine learning model configuration data. For example, the system may determine that the medical billing classification prediction machine learning model receives as input data N values corresponding to N features. The N features may correspond to N tokens representing N words and/or sub-words. The system may generate M sequences by dividing a total number of tokens (i.e., words and sub-words) T by N, where T/N=M, if the number of features N divides evenly into the number of tokens T, and T/N=M−1, if the number of features N divides into the number of tokens T with a remainder. For example, if the number of tokens that a medical billing classification prediction machine learning model is configured to receive is N=512, then the system generates one sequence if T is less than or equal to 512, two sequences if Tis greater than 512 and less than or equal to 1024, etc.

In one embodiment, the system selects a number of sequences and a size of the sequences based on machine learning model configuration data, such as the size and type of input data the machine learning model is configured to receive. Additionally, or alternatively, the system may generate a number of tokens or modify a number of generated tokens based on the machine learning model configuration data. For example, if the number of features N divides into the number of tokens T with a remainder, the system may generate a number of additional sub-words equal to the remainder. Alternatively, if the remainder is below a threshold, the system may remove a number of sub-words equal to the remainder from the set of tokens.

The system applies a trained machine learning model to a sequence of tokens to generate a set of probabilities for a set of medical billing classification codes (Operation 208). In one embodiment, the trained machine learning model includes a transformer-type machine learning model, such as a bidirectional encoder representation from transformers (BERT) type model. The machine learning model may be fine-tuned on a medical-notation type dataset. The BERT type model generates embedding vectors representing the tokens that correspond to the set of words and sub-words in the sequence. The machine learning model may be further fine-tuned with one or more classification/prediction layers added to the end layer of the BERT type model to generate the set of probabilities for the medical billing classification codes.

In one embodiment, the machine learning model generates a probability value for each medical billing classification code in a set of medical billing classification codes. For example, the system may be configured to generate probability values for the 10 most frequently used medical billing classifications.

The system determines if additional sequences exist from among the M sequences (Operation 210). If an additional sequence exists (210—Yes), the system applies the trained machine learning model to the additional sequence (Operation 208). The system repeats operations 208 and 210 until the system has generated sets of medical billing classification code/probability value pairs for every sequence M.

Based on the sets of sequence probability values for the respective sequences, the system selects one or more predicted medical billing classification codes for the medical notation data (Operation 212). In one or more embodiments, the system applies a mathematical or logical algorithm to the sets of probability values for the set of sequences associated with the medical notation data to select a predicted medical billing classification code for the medical notation data. For example, the system may calculate the mean value for each probability value across the set of sequences. The system may select the medical billing classification code corresponding to the highest mean probability value as the predicted medical billing classification code for the medical notation data. Alternatively, the system may select the medical billing classification code associated with the highest overall probability value as the predicted medical billing classification code for the medical notation data. According to yet another alternative, the system may select the medical billing classification code associated with the highest cumulative probability value (i.e., the sum of the probability values across the set of sequences is the highest) as the predicted medical billing classification code for the medical notation data.

In one or more embodiments, the system ranks the medical billing classification codes according to their probability values. The system may create a predicted medical billing classification code grouping based on the ranked medical billing classification codes. For example, the system may create a grouping of the three highest-ranked medical billing classification codes, according to their mean probability values.

The system applies the predicted medical billing classification code(s) to a medical billing model to generate a predicted medical billing value for a patient visit (Operation 214). For example, the system may store a mapping to medical billing classification codes to billing coefficients. The medical billing model may multiply a base value to variable billing coefficients that differ based on the medical billing classification codes. In one example, the system generates a predicted medical billing value by multiplying the base value to a set of multiple coefficients corresponding to a set number of predicted medical billing classification codes with the highest probability values. For example, the system may identify the three medical billing classification codes having the highest mean probability values across a set of sequences. The system may multiply the base billing value by three coefficient values corresponding to the three medical billing classification codes. The system may further multiply the respective coefficient values by weight values corresponding to the respective probability values for the medical billing classification codes.

A medical services entity may use the predicted medical billing value for the services to predict the most likely amount the entity will be able to bill for treating a patient. The medical services entity may employ the above operations prior to discharging the patient, such as within the first 24 or 48 hours of patient intake. By employing the operations for predicting medical billing, the medical services entity may generate operational forecasts and adjust treatment plans.

### 4. Training a Machine Learning Model to Generate Probability Values for Medical Billing Classification Codes

A detailed example is described below for purposes of clarity. Components and/or operations described below should be understood as one specific example that may not be applicable to certain embodiments. Accordingly, components and/or operations described below should not be construed as limiting the scope of any of the claims.

A system obtains a pre-trained machine learning model (Operation 302). In one embodiment, the pre-trained machine learning model is an encoder-only type machine learning model. The encoder-only type ML model is trained on a dataset that includes a broad vocabulary to learn relationships among words and grammatical rules. The pre-trained ML model is trained to receive a sequence of tokens that represent words and sub-words as input data and to generate an embedding representing the sequence as output data. The embedding is a multi-dimensional numerical vector. In example embodiments, the pre-trained ML model is a BERT type model or a ClinicalBERT type model.

The system creates a classification head by attaching an additional neural network layer to the output of the pre-trained ML model (Operation 304). Adding the classification head results in generating a different type of output data from the pre-trained ML model. While the pre-trained ML model is configured to receive sequences of tokens as input data and generate embeddings for the sequences as output data, the classification head is configured to receive the embeddings from the pre-trained ML model as input data and generate probability values for a set of medical billing classification codes as output data.

The system freezes the parameters of the pre-trained ML model (Operation 306). The offsets and coefficients of the pre-trained ML model are set at their pre-trained values to prevent the parameters from changing in subsequent training of the fine-tuned ML model including the classification head.

The system trains the medical billing codes classification prediction model that includes the pre-trained model and the classification head with medical billing classification datasets to generate probabilities for a set of medical billing classification codes (Operation 308). During training of the medical billing probability prediction model, the parameters of the pre-trained ML model remain frozen while the system modifies the parameters of the neurons that make up the classification head.

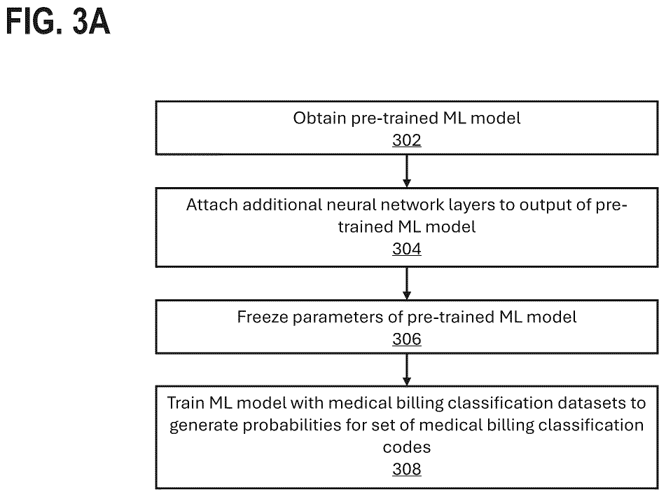

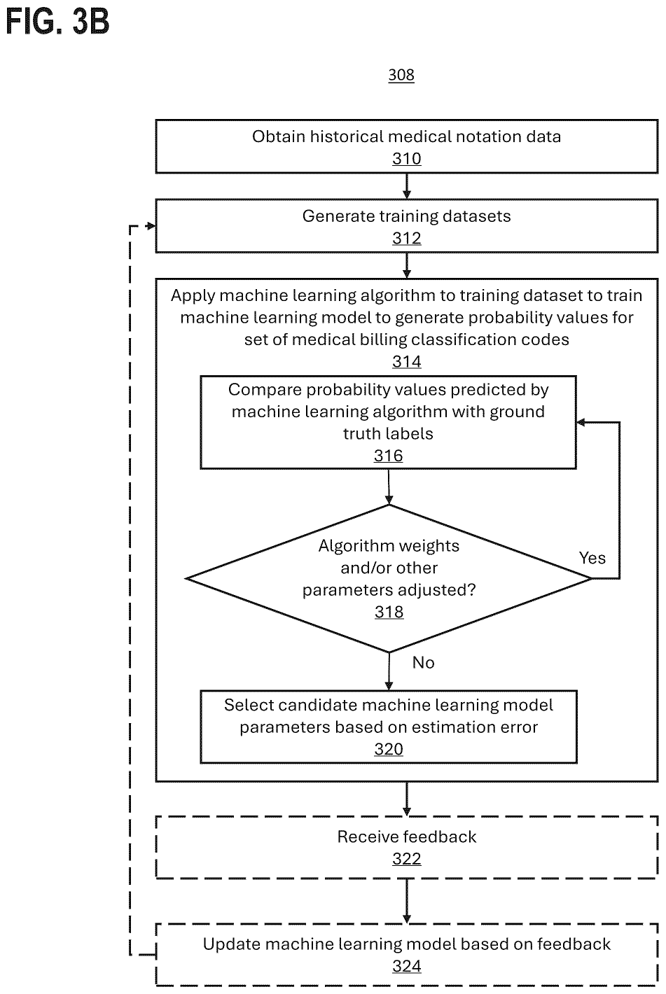

FIG. 3B describes the training of the medical billing probability prediction model in further detail. In one or more embodiments, a system (e.g., one or more components of system 100 illustrated in FIG. 1) obtains historical medical notation data (Operation 310). Obtaining the historical medical notation data may include obtaining any of the following: patient intake surveys, medical provider test data, vitals data, physician consultation notes, physician diagnosis notes, other healthcare service records, charges assigned to medical services, and medical billing classification codes assigned to patient visits.

The system uses the historical medical notation data to generate a set of training data (Operation 312). The set of training data includes, for a particular set of medical notation data, at least one classification label. For example, after a patient is discharged, a medical provider may assign a medical billing classification code to a claim associated with the patient's treatment. The system may generate the set of training data by omitting the portion of the medical notation data after a predefined period of time, such as 24 hours or 48 hours, from the training data. In addition, the system may further omit from the training data any medical notation data that includes the patient discharge within the predefined period of time.

As an example, if the predefined period of time is 48 hours, the system omits from the training data any medical notation data for which the patient was discharged within 48 hours of being admitted. In addition, the system omits from the training data any medical notation data generated more than 48 hours after the patient was admitted. The resulting sets of training data may include the same or similar sets of words associated with different medical billing classification codes. For example, a set of symptoms including fever, sore throat, and upset stomach may correspond to multiple medical billing classification codes. A medical provider may not narrow down the diagnosis and corresponding medical billing classification code until obtaining certain test results after 48 hours has elapsed. As a result, the machine learning model may learn that the same or similar sets of input data (e.g., tokens corresponding to medical notation data) may be associated with multiple different medical billing classification codes. The system trains the ML model to generate probability values for the different medical billing classification codes.

According to one embodiment, the system generates probability values as classification labels for the training data. The system may identify, for sets of tokens corresponding to different sets of medical notation data, the probability that the set of tokens is associated with different medical billing classification codes. Alternatively, the system generates medical billing classification codes as classification labels for the training data. Over the course of training, the ML model learns to generate the probability values associated with the different medical billing classification codes.

According to one embodiment, the system obtains the historical medical notation data and the training data set from a data repository storing labeled data sets. The training data set may be generated and updated by a healthcare service provider or organization. Alternatively, the training data set may be generated and maintained by a third party. According to one embodiment, the system generates the labeled set of data by parsing documents and generating labels based on parsed values in the documents. According to an alternative embodiment, one or more users generate labels for a data set.

In some embodiments, generating the training data set includes generating a set of feature vectors for the labeled examples. A feature vector, for example, may be n-dimensional, where n represents the number of features in the vector. The number of features that are selected may vary depending on the particular implementation. The features may be curated in a supervised approach or automatically selected from extracted attributes during model training and/or tuning. Example features include information about a healthcare provider that provided a healthcare service to a patient, geographic information about where a healthcare service was provided (e.g., a facility or a region), temporal information about when a service was provided (e.g., date and time), categorical information about what type of healthcare service was provided (e.g., out-patient service, regularly-scheduled check-up, overnight treatment, mental health services, surgical procedures, emergency services), and the cost to provide the healthcare service. In some embodiments, a feature within a feature vector is represented numerically by one or more bits. The system may convert categorical attributes to numerical representations using an encoding scheme, such as one-hot encoding, label encoding, and binary encoding. One-hot encoding creates a unique binary feature for each possible category in an original feature. In one-hot encoding, when one feature has a value of 1, the remaining features have a value of 0. For example, if a type of healthcare service has ten different categories, the system may generate ten different features of an input data set. When one category is present (e.g., value “1”), the remaining features are assigned a value “0.” According to another example, the system may perform label encoding by assigning a unique numerical value to each category. According to yet another example, the system performs binary encoding by converting numerical values to binary digits and creating a new feature for each digit.

The system applies a machine learning algorithm to the training data set to train the machine learning model (Operation 314). For example, the machine learning algorithm may analyze the training data set to train neurons of a neural network in the classification head of the ML model with particular weights and offsets to associate particular medical notation data with particular medical billing classification codes and/or medical billing classification code probability values. The system trains the neurons of the neural network of the classification head without modifying neurons of the pre-trained ML model.

In some embodiments, the system iteratively applies the machine learning algorithm to a set of input data to generate an output set of labels, compares the generate labels to pre-generated labels associated with the input data, adjusts weights and offsets of the algorithm based on an error, and applies the algorithm to another set of input data.

In some embodiments, the system compares the probability values estimated through the one or more iterations of the machine learning model algorithm with ground truth labels to determine an estimation error (Operation 316). The system may perform this comparison for a test set of examples that may be a subset of examples in the training dataset that were not used to generate and fit the candidate models. The total estimation error for a particular iteration of the machine learning algorithm may be computed as a function of the magnitude of the difference and/or the number of examples for which the estimated label was wrongly predicted.

In some embodiments, the system determines whether or not to adjust the weights and/or other model parameters based on the estimation error (Operation 318). Adjustments may be made until a candidate model that minimizes the estimation error or otherwise achieves a threshold level of estimation error is identified. The process may return to Operation 318 to make adjustments and continue training the machine learning model.

In some embodiments, the system selects machine learning model parameters based on the estimation error meeting a threshold accuracy level (Operation 320). For example, the system may select a set of parameter values for a machine learning model based on determining that the trained model has an accuracy level for predicting labels for medical claims of at least 98%.

In some embodiments, the system trains a neural network of the classification head using backpropagation without applying the backpropagation to the pre-trained ML model. Backpropagation is a process of updating cell states in the neural network based on gradients determined as a function of the estimation error. With backpropagation, nodes are assigned a fraction of the estimated error based on the contribution to the output and adjusted based on the fraction. In recurrent neural networks, time is also factored into the backpropagation process. For example, a set of medical notation data may be processed as a separate discrete instance of time. For instance, an example may include medical notation data c₁, c₂, and c₃ that correspond to times t, t+1, and t+2, respectively. Backpropagation through time may perform adjustments through gradient descent starting at time t+2 and moving backward in time to t+1 and then to t. Further, the backpropagation process may adjust the memory parameters of a cell such that a cell remembers contributions from previous expenses in the sequence of expenses. For example, a cell computing a contribution for e₃ may have a memory of the contribution of e₂, which has a memory of e₁. The memory may serve as a feedback connection such that the output of a cell at one time (e.g., t) is used as an input to the next time in the sequence (e.g., t+1). The gradient descent techniques may account for these feedback connections such that the contribution of one medical claim to a cell's output may affect the contribution of the next medical claim in the cell's output. Thus, the contribution of c₁ may affect the contribution of c₂, etc.

In embodiments in which the machine learning algorithm is a supervised machine learning algorithm, the system may optionally receive feedback on the various aspects of the analysis described above (Operation 322). For example, the feedback may affirm or revise labels generated by the machine learning model. The machine learning model may indicate that a particular set of medical notation data has a 0.9 probability of being associated with a particular medical billing classification code. The system may receive feedback indicating that the particular medical billing classification code should instead be associated with a 0.6 probability value. Based on the feedback, the machine learning training set may be updated (Operation 324), thereby improving its analytical accuracy. Once updated, the system may further train the machine learning model by optionally applying the model to additional training data sets.

### 4. Example Embodiment

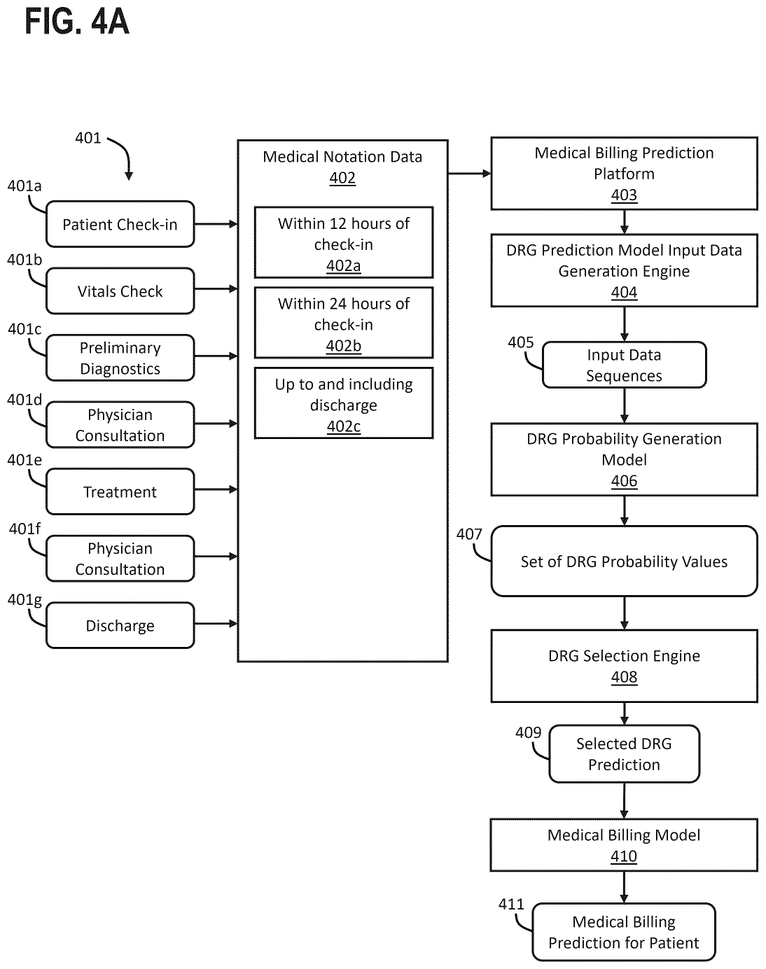

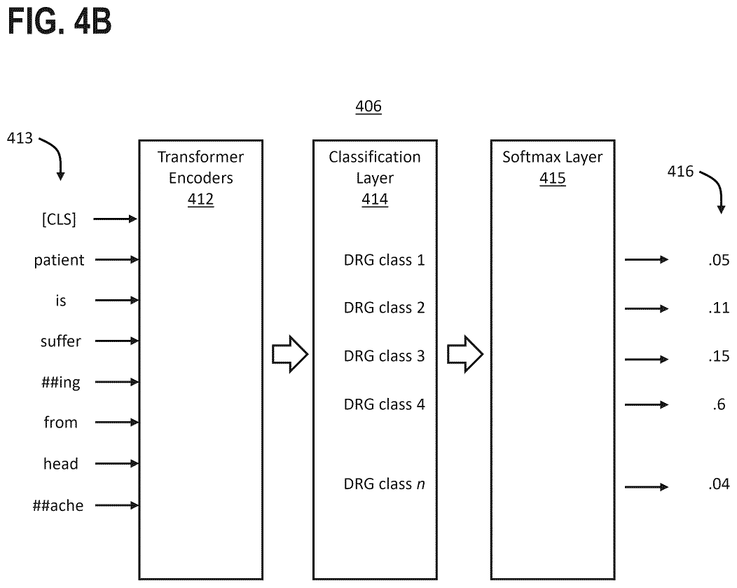

For purposes of clarity, a detailed example is described in FIGS. 4A and 4B below. Components and/or operations described below should be understood as one specific example that may not be applicable to certain embodiments. Accordingly, components and/or operations described below should not be construed as limiting the scope of any of the claims.

Referring to FIG. 4A, a patient visit may include a set of events 401 in which a medical provider receives or generates medical notation data 402. For example, a user may fill out a symptom and medical history form at patient check-in 401a. A nurse may record a patient's vitals information, such as blood pressure and temperature, at a vitals check 401b. A physician may meet with a patient and generate a preliminary diagnosis 401c. The physician may generate patient data during a physician consultation 401d. Medical staff may record treatment data, including types of treatments and results, during a patient treatment phase 401e. The physician may generate additional medical data at additional physician consultations 401f post-treatment. Medical staff may generate additional data, such as a patient condition and a prescribed course of treatment, when the patient is discharged 401g.

The medical billing prediction platform 403 obtains medical notation data 402 from prior to the patient's discharge to predict DRG classification codes and medical billing for the patient's future services and/or treatment. For example, the medical billing prediction platform 403 may be configured to generate a medical billing prediction based on a set of medical notation data 402a generated (a) within 12 hours of the patient's check-in and (b) prior to the patient's discharge. As another example, the medical billing prediction platform 403 may be configured to generate a medical billing prediction based on a set of medical notation data 402b generated (a) within 24 hours of the patient's check-in and (b) prior to the patient's discharge. The medical billing prediction platform 403 may be configured to refrain from using medical notation data 402c generated at the time a patient is discharged or after a predefined time (e.g., 12 hours or 24 hours) after a patient's check-in to generate medical billing predictions. For example, when the medical billing prediction platform 403 is configured to use medical notation data 402 from within 12 hours of a patient's check-in, the medical billing prediction platform uses neither any medical notation data 402 generated after 12 hours have elapsed from the patient's check in nor any medical notation data generated at the time of the patient's discharge.

The DRG prediction model input data generation engine 404 (a) processes the medical notation data to clean the text, (b) generates sub-words from words in the text, (c) generates tokens representing the words and sub-words, and (d) divides the tokens representing the words and sub-words generated from the medical notation data 402 into sequences 405 based on configuration data of the DRG probability generation model 406. For example, if the DRG prediction model input data generation engine 404 determines the DRG probability generation model 406 has an input feature limit of 500 values, and if the set of tokens representing the words and sub-words of the medical notation data 402a equals 2050 tokens, the DRG prediction model input data generation engine 404 may generate five sequences of tokens—four sequences of 500 tokens and one sequence of 50 tokens plus 450 nonce or filler tokens that may not carry meaning but that fill out the set of 500 tokens.

The DRG probability generation model 406 generates sets of DRG probability values 407 based on the set of sequences 405 corresponding to the medical notation data 402a. FIG. 4B illustrates an example of the implementation of the DRG probability generation model 406.

A sequence 413, that may be among the set of sequences 405 shown in FIG. 4A, is input to a set of transformer encoders 412. In FIG. 4B, the sequence is represented as words and sub-words for the purposes of description. However, in practice, the sequence may include a set of tokens, such as numerical values, representing the words and sub-words. In the example illustrated in FIG. 4B, “patient,” “is,” and “from” are words, and “suffer,” “##ing,” “head,” and “##ache” are sub-words generated from the words “suffering” and “headache” in the medical notation data 402a.

The set of transformer encoders 412 are part of a pre-trained machine learning model. In the example of FIGS. 4A and 4B, the pre-trained machine learning model is a ClinicalBERT type model. The ClinicalBERT type model is a Bidirectional Encoder Representations from Transformers (BERT) type model that, in addition to being trained on a broad corpus of text content, is further trained on clinical text content.

A classification layer 414 receives an embedding from the transformer encoders 412. The embedding represents the sequence 413. The classification layer generates a set of values representing probabilities associated with DRG classes 1-n. A Softmax layer 415 receives the probability values and converts the values to a value between 0 and 1, such that the sum of the probability values equals 1. The Softmax layer 415 outputs the probability values 416.

The system provides each input data sequence 405 corresponding to the medical notation data 402a to the DRG probability generation model 406 to generate the set of DRG probability values 407. For example, while FIG. 4B illustrates the generation of one set of probability values 416 corresponding to one sequence 413, the set of DRG probability values 407 is made up of multiple sets of probability values generated based on multiple sequences.

The DRG selection engine 408 selects a DRG prediction 409 based on the set of DRG probability values 407. For example, the set of DRG probability values may be visualized, for purposes of description, as a table. The far-left column of the table may specify sequence names (e.g., sequ1, sequ2, . . . , sequn). The header of the table may specify DRG classes (e.g., DRG 345, DRG 346, . . . , DRG n). The rows in the table specify the probability values for each of the DRG classes in the header. The DRG selection engine 408 calculates the mean probability for each DRG class across the sequences (e.g., sequ1, sequ2, . . . sequn). The DRG selection engine 408 selects the DRG class that corresponds to the highest mean probability as the selected DRG prediction 409 for the medical notation data 402a.

The system provides the DRG classification code prediction 409 for the medical notation data 402a to a medical billing model 410 to generate a medical billing prediction 411 for a patient. The medical billing model 410 generates the prediction based on a formula that includes the following: the predicted DRG classification code, a standardized amount associated with the healthcare provider generating the medical notation data, a cost of living adjustment based on a geographic location of a healthcare provider, a patient's age, a number of diagnoses, a predicted length of stay, and if a healthcare provider is a teaching hospital.

The healthcare provider uses the medical billing prediction, generated from the DRG classification code prediction based on the medical notation data 402a from the 12 hours after a patient checked in, to predict longer-term billing for the patient and generate or modify components of a treatment plan for the patient.

### 5. Computer Networks and Cloud Networks

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis.

### 7. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

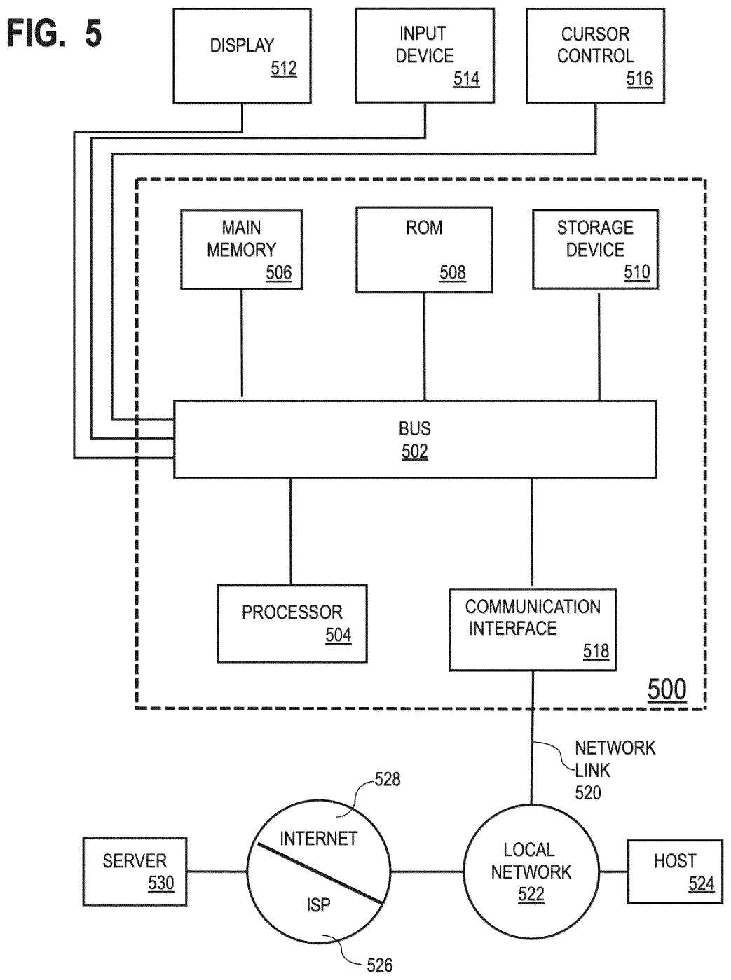

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general-purpose microprocessor.

Computer system 500 also includes a main memory 506, such as a random-access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or a Solid-State Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid-state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the worldwide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 8. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
receiving medical notation data for a target patient corresponding to at least one of: (a) a discussion between a physician and a patient, or (b) a physician-generated medical diagnosis of the patient;
partitioning the medical notation data into a plurality of sequences;
for each particular sequence of the plurality of sequences comprised in the medical notation data: applying a trained machine learning model to the particular sequence to determine a probability value for each of a plurality of medical billing classification codes;
for each particular medical billing classification code of the plurality of medical billing classification codes, determining a probability of the target patient being associated with the particular medical billing classification code as a function of the probability values computed for the particular medical billing classification code based on each of the plurality of sequences comprised in the medical notation data;
based on the respective probability of each medical billing classification code of the plurality of medical billing classification codes, selecting a first medical billing classification code of the plurality of medical billing classification codes as a predicted medical billing classification code for the patient; and
storing the predicted medical billing classification code for the patient.

2. The non-transitory computer readable media of claim 1, wherein partitioning the medical notation data into the plurality of sequences is based on model configuration data associated with the trained machine learning model.

3. The non-transitory computer readable media of claim 1, wherein computing the probability of the target patient being associated with the particular medical billing classification code as the function of the probability values computed for the particular medical billing classification code based on each of the plurality of sequences comprises computing a weighted average of the probability values respectively associated with the plurality of sequences for the particular medical billing classification code based on respective characteristics of the plurality of sequences.

4. The non-transitory computer readable media of claim 1, wherein the operations further comprise:
identifying a subset of medical billing classification codes, from among the plurality of medical billing classification codes, based on determining the subset of medical billing classification codes corresponds to a number, n, of medical billing classification codes associated with the n-highest probability values; and
predicting a medical billing value associated with the target patient based on a combination of the n medical billing classification codes.

5. The non-transitory computer readable media of claim 1, wherein the operations comprise:
training the machine learning model to determine the probability value for each of the plurality of medical billing classification codes at least by:
obtaining a pre-trained encoder-only type machine learning model;
generating a neural network layer to receive an output from the pre-trained encoder-only type machine learning model;
obtaining a plurality of training data sets, a training data set of the plurality of training data sets comprising:
medical notation data associated with medical treatment of a historical patient; and
at least one label associated with a historical medical billing classification code assigned to the historical patient; and
applying the plurality of training data sets to a machine learning algorithm to determine first parameters for the trained machine learning model at least by:
freezing second parameters of the pre-trained encoder-only type machine learning model while modifying third parameters of neural network layer based on an error function.

6. The non-transitory computer readable media of claim 1, wherein partitioning the medical notation data into the plurality of sequences comprises:
determining an input feature limit for the trained machine learning model;
determining a number of tokens in a set of tokens generated by converting the medical notation data into tokens exceeds the input feature limit; and
dividing the set of tokens exceeding the input feature limit into the plurality of sequences that do not exceed the input feature limit.

7. The non-transitory computer readable media of claim 1, wherein the operations further comprise:
providing the medical notation data to a decoder-only type machine learning model to generate a natural language summary of the medical notation data;
wherein partitioning the medical notation data into the plurality of sequences comprises:
generating a set of tokens from the natural language summary; and
partitioning the set of tokens into the plurality of sequences.

8. A method comprising:
receiving medical notation data for a target patient corresponding to at least one of: (a) a discussion between a physician and a patient, or (b) a physician-generated medical diagnosis of the patient;
partitioning the medical notation data into a plurality of sequences;
for each particular sequence of the plurality of sequences comprised in the medical notation data: applying a trained machine learning model to the particular sequence to determine a probability value for each of a plurality of medical billing classification codes;
for each particular medical billing classification code of the plurality of medical billing classification codes, determining a probability of the target patient being associated with the particular medical billing classification code as a function of the probability values computed for the particular medical billing classification code based on each of the plurality of sequences comprised in the medical notation data;
based on the respective probability of each medical billing classification code of the plurality of medical billing classification codes, selecting a first medical billing classification code of the plurality of medical billing classification codes as a predicted medical billing classification code for the patient; and
storing the predicted medical billing classification code for the patient,
wherein the method is performed by at least one device including a hardware processor.

9. The method of claim 8, wherein partitioning the medical notation data into the plurality of sequences is based on model configuration data associated with the trained machine learning model.

10. The method of claim 8, wherein computing the probability of the target patient being associated with the particular medical billing classification code as the function of the probability values computed for the particular medical billing classification code based on each of the plurality of sequences comprises computing a weighted average of the probability values respectively associated with the plurality of sequences for the particular medical billing classification code based on respective characteristics of the plurality of sequences.

11. The method of claim 8, further comprising:
identifying a subset of medical billing classification codes, from among the plurality of medical billing classification codes, based on determining the subset of medical billing classification codes corresponds to a number, n, of medical billing classification codes associated with the n-highest probability values; and
predicting a medical billing value associated with the target patient based on a combination of the n medical billing classification codes.

12. The method of claim 8, further comprising:
training the machine learning model to determine the probability value for each of the plurality of medical billing classification codes at least by:
obtaining a pre-trained encoder-only type machine learning model;
generating a neural network layer to receive an output from the pre-trained encoder-only type machine learning model;
obtaining a plurality of training data sets, a training data set of the plurality of training data sets comprising:
medical notation data associated with medical treatment of a historical patient; and
at least one label associated with a historical medical billing classification code assigned to the historical patient; and
applying the plurality of training data sets to a machine learning algorithm to
determine first parameters for the trained machine learning model at least by:
freezing second parameters of the pre-trained encoder-only type machine learning model while modifying third parameters of neural network layer based on an error function.

13. The method of claim 8, wherein partitioning the medical notation data into the plurality of sequences comprises:
determining an input feature limit for the trained machine learning model;
determining a number of tokens in a set of tokens generated by converting the medical notation data into tokens exceeds the input feature limit; and
dividing the set of tokens exceeding the input feature limit into the plurality of sequences that do not exceed the input feature limit.

14. The method of claim 8, further comprising:
providing the medical notation data to a decoder-only type machine learning model to generate a natural language summary of the medical notation data,
wherein partitioning the medical notation data into the plurality of sequences comprises:
generating a set of tokens from the natural language summary; and
partitioning the set of tokens into the plurality of sequences.

15. A system comprising:
at least one device including a hardware processor;
the system being configured to perform operations comprising:
receiving medical notation data for a target patient corresponding to at least one of: (a) a discussion between a physician and a patient, or (b) a physician-generated medical diagnosis of the patient;
partitioning the medical notation data into a plurality of sequences;
for each particular sequence of the plurality of sequences comprised in the medical notation data: applying a trained machine learning model to the particular sequence to determine a probability value for each of a plurality of medical billing classification codes;
for each particular medical billing classification code of the plurality of medical billing classification codes, determining a probability of the target patient being associated with the particular medical billing classification code as a function of the probability values computed for the particular medical billing classification code based on each of the plurality of sequences comprised in the medical notation data;
based on the respective probability of each medical billing classification code of the plurality of medical billing classification codes, selecting a first medical billing classification code of the plurality of medical billing classification codes as a predicted medical billing classification code for the patient; and
storing the predicted medical billing classification code for the patient.

16. The system of claim 15, wherein partitioning the medical notation data into the plurality of sequences is based on model configuration data associated with the trained machine learning model.

17. The system of claim 15, wherein computing the probability of the target patient being associated with the particular medical billing classification code as the function of the probability values computed for the particular medical billing classification code based on each of the plurality of sequences comprises computing a weighted average of the probability values respectively associated with the plurality of sequences for the particular medical billing classification code based on respective characteristics of the plurality of sequences.

18. The system of claim 15, wherein the operations further comprise:
identifying a subset of medical billing classification codes, from among the plurality of medical billing classification codes, based on determining the subset of medical billing classification codes corresponds to a number, n, of medical billing classification codes associated with the n-highest probability values; and
predicting a medical billing value associated with the target patient based on a combination of the n medical billing classification codes.

19. The system of claim 15, wherein the operations comprise:
training the machine learning model to determine the probability value for each of the plurality of medical billing classification codes at least by:
obtaining a pre-trained encoder-only type machine learning model;
generating a neural network layer to receive an output from the pre-trained encoder-only type machine learning model;
obtaining a plurality of training data sets, a training data set of the plurality of training data sets comprising:
medical notation data associated with medical treatment of a historical patient; and
at least one label associated with a historical medical billing classification code assigned to the historical patient; and
applying the plurality of training data sets to a machine learning algorithm to
determine first parameters for the trained machine learning model at least by:
freezing second parameters of the pre-trained encoder-only type machine learning model while modifying third parameters of neural network layer based on an error function.

20. The system of claim 15, wherein partitioning the medical notation data into the plurality of sequences comprises:
determining an input feature limit for the trained machine learning model;
determining a number of tokens in a set of tokens generated by converting the medical notation data into tokens exceeds the input feature limit; and
dividing the set of tokens exceeding the input feature limit into the plurality of sequences that do not exceed the input feature limit.
